(19) United States
(12) Patent Application Publication
(10) Pub. No.: US 2016/0030630 A1
(43) Pub. Date: Feb. 4, 2016

(54) ANTI-FUNGUS METHODS AND KIT FOR TREATMENT OF FUNGAL NAIL INFECTIONS

(71) Applicant: Scot Douglas Kingsbury, Huntington Station, NY (US)

(72) Inventor: Scot Douglas Kingsbury, Huntington Station, NY (US)

(21) Appl. No.: 14/446,943

(22) Filed: Jul. 30, 2014

(51) Int. Cl. A61L 26/00

(52) U.S. Cl. CPC A61L 26/0066; A61L 26/00; A61L 26/0057

(57) ABSTRACT

The present invention relates generally to methods and related kit to maximize the effectiveness of anti-fungus medicines including Vicks Vapor Rub and to mitigate the spread of infection to other toes or fingers and to other people. The kit consists of part of disposable nitrile glove or the like, anti-fungus medicine such as topical drug, and a tape. Barrier-inverting method and rolling-up method have been disclosed on how to apply the kit to the infected areas.

### FIELD OF THE INVENTION

The present invention relates generally to methods and related kit to maximize the effectiveness of the anti-fungal properties of anti-fungus medicines including Vicks Vapor Rub and to mitigate the spread of infection to other toes or fingers and to other people from shower or other situations. The objective of this invention is to bathe the infected toe(s) or finger(s) continuously in medicine (under a vapor barrier) until the infection is defeated. More specifically, the invention relates to a kit consisting of part of disposable nitrile glove, a topically active anti-fungal agent, and a tape. Moreover, two methods have been disclosed on how to apply the kit to the infected areas.

### BACKGROUND OF THE INVENTION

Fungal nail disease is the most commonly occurring nail disorder encountered in primary care. Current estimates indicate that nearly 11 million Americans are affected by fungal nail disease. It can affect toenails, fingernails or both and is usually caused by infectious organisms known as dermatophytes. Fungal nail disease causes discoloration and thickening of the nail, accumulation of debris under the nail and, in severe cases, detachment of the nail plate from the nail bed. Toenails are the primary site of infection. Fungal nail disease can affect people of any age, gender and race and may cause discomfort and embarrassment due to the appearance of the nails.

It is estimated that up to 35 million Americans have onychomycosis, a condition relating to fungal infections of the toenails or fingernails. Onychomycosis is often caused by yeast, derrnatophytes, or other molds, and represents approximately 50% of all nail disorders. Toenail infection accounts for approximately 80% of onychomycosis incidence, while fingernails are affected in about 20% of the cases. Dermatophytes are the most frequent cause of nail plate invasion, particularly in toenail onychomycosis. Onychomycosis caused by a derrnatophyte is termed generally termed tineaunguium. Trichophylon Rubrumis by far the most frequently isolated derrnatophyte, followed by T. Menlagrophyles. Distal subungual onychomycosis is the most common presentation of tinea unguium, with the main site of entry through the hyponychium (the thickened epidermis underneath the free distal end of a nail) progressing in time to involve the nail bed and the nail plate. Discoloration, onycholysis, and accumulation of subungual debris and nail plate dystrophy characterize the disease. The disease adversely affects the quality of life of its victims, with subject complaints ranging from unsightly nails and discomfort with footwear, to more serious complications including secondary bacterial infections.

About 7% of Americans suffer from onychomycosis, a fungal infection of the nail, particularly the toenail. More than 90 percent of cases are caused by one of two pathogens: Trichophylon Rubrum or Trichophylon Menlagrophyles. Factors that have an important effect on the development of onychomycosis include increasing age; genetic susceptibility; and the presence of certain disease states such as diabetes, acquired immunodeficiency syndrome, or peripheral arterial disease. Of particular concern are diabetics who are nearly three times more likely to develop onychomycosis than nondiabetics. It has been reported that up to one third of diabetics develop nail fungus. In diabetics, slow healing, particularly in the feet, is common and foot infections of all types can be difficult to treat and may end in amputation.

Onychomycosis is a medical condition with cosmetic impact because it disfigures the nails. Nails can become thick, discolored, loose, brittle, hard, yellow and painful. Once nails are infected, even if healthy nail is grown out, the susceptibility to infection may remain and the condition can relapse. Furthermore, healthy nails can be re-infected.

Onychomycosis (tinea unguium) is defined as a localized infection of the nail or nail bed primarily caused by a pathogenic fungi; however, yeasts and molds can also cause infection. This disease is not life threatening, however, it can cause inconvenience, pain and discomfort to the individual and serve as a reservoir for infection. Infection can usually be determined by thickened, yellow or brown discolored, friable nail plates.

Onchyomycosis affects up to 30% of the population by age 60. The most common dermatophytes are Trichophyton rubrum and Trichophyton mentagrophytes. Although there are numerous topical agents on the market, none have been found to be satisfactory. Consequently, emphasis in research studies has focused on oral treatments. Fungal infections of the fingernails or toenails are caused most commonly by dermatophytes (Tricophyton rubrum, T mentagrophytes, Microsporum canis, Epidermophyton F loccosum and E. stocka'ale) which represent about 90% of the infection. Yeasts (Candida albicans, C. parapsilosis. and C. krusei) and nondermatophyte molds (Scytalidium hyalinum, S. dimidiatum, Fusarium oxysporum, E moniliforme, Acremonium chrysogenum, A. strictum, Aspergillus terreus, and Scopulariopsis brevicaulis) are responsible for 7% and 3% of infections, respectively. These organisms infect the stratum corneum of the skin, hair and nails Immunodeficient patients, such as those with AIDS, the nail infection can be severe. Also, diabetics are known to be predisposed to certain cutaneous diseases but their frequency of infection is still not clear.

Fungal nail infections may be difficult to treat and it is common for the fungus to return. Over-the-counter creams and ointments generally do not help treat this condition and prescription antifungal medicines taken by mouth may help clear the fungus in about 50% of patients. However, such medicines can cause side effects or may interfere with other medications and some of the oral medications used to treat fungal infections of the nail can harm the liver. In some cases, the health care provider may remove the nail. Nails grow slowly. Even if treatment is successful, a new, clearer nail may take up to a year to grow in. The fungal nail infection is cured by the growth of new, non-infected nails.

While there are topically applied reagents that effectively control fungal growth on skin (e.g., cyclopirox), getting the reagent to thoroughly contact the fungus throughout the nail and nail bed has long been the challenge.

Based on the limitations of the current onychomycosis therapies, there is a pronounced need in the art for new treatments for fungal infections associated with fingernails and toenails.

Treatments with topical creams are not as effective as oral treatments and prolonged periods of application are required. Orally administered drugs that are available on the market also suffer from various drawbacks. Some are not very effective in controlling the fungal infection, some produce unwanted side effects, and all are quite expensive when compared to topical drugs.

It's unquestionable that a successful, safe, and noninvasive topical treatment for nail infections is a long-felt and unsolved need. This is an undeniable fact that is well-understood and accepted by those skilled in the art. The problem lies in transporting the anti-infective agents through the nail and into the nail bed, as well as other regions in and around the nail, to reach all sites and sources of the infection.

One of skill will appreciate that research has long focused on developing a topical reagent which both penetrates the nail and destroys the fungus. Unfortunately, the research has not produced a solution. Ciclopirox, azoles, and other topical antifungal agents have shown some positive results, but the cure rate is low, and at best around 20% after a year of treatment with ciclopirox, for example. Most cure rates are typically lower.

Onychomycosis never resolves spontaneously and recurrence after treatment is common. Treatment is difficult because of the unique properties of the nail unit. Thus, an effective antifungal agent must enter the affected tissue and persist there in high concentrations. The existing therapies must be continuous and used until the infected nail grows out and even after this lengthy treatment low cure rates and quick relapse times are common.

Toenail fungus usually gains entry to the toenail bed when there has been an injury to the nail, such as a crack or a split. Once the fungus has entered, the warm, dark environment that it finds in the shoes and socks encourages it to grow and spread. According to the Foot Health Network, the standard treatment for toenail fungus involves prescription medication or partial to full nail removal. Many people want to avoid harsh medications and painful treatment and are turning to a remedy for toenail fungus you may not suspect: Vicks Vapor Rub.

It seems as though Vicks Vapor Rub has been a cure for almost everything, from coughs to chest congestion to stuffy noses. Now Vicks is emerging as a cure for nail fungus. What may have started as an accidental home remedy now has a medical study to back it up. Researchers at Michigan State University studied the effects of Meijer medicated chest rub on toenail fungus and found the ratio of camphor, eucalyptus oil and menthol to be effective in treating the fungus. While the brand was Meijer, it has the exact same ratio of ingredients that Vicks Vapor Rub has, which explains why people are having so much success with Vicks.

Accordingly, those skilled in the art of treating nail infections, and the patients suffering such nail infections, will appreciate a successful, safe, and non-invasive topical treatment for nail infections. The present invention provides such a method of treatment that (i) is topical and safe; (ii) does not require oral or systemic administration of drugs; (iii) is safer for patients that may be intolerant to systemic drug delivery; (iv) is several times faster than existing topical treatments; (v) does not require removal of the nail; and (vi) does not require the patient to do without the presence of a nail for the year or so required to grow a new nail.

### SUMMARY OF THE INVENTION

The present invention relates generally to methods and related kit to maximize the effectiveness of the anti-fungal properties of anti-fungus medicines including Vicks Vapor Rub and to mitigate the spread of infection to other toes or fingers and to other people from shower or other situations. The objective of this invention is to bathe the infected toe(s) or finger(s) continuously in medicine (under a vapor barrier) until the infection is defeated. More specifically, the invention relates to a kit consisting of part of disposable nitrile glove or the like, anti-fungus medicine such as topical drug, and a tape. Moreover, two methods have been disclosed on how to apply the kit to the infected areas. One method is a barrier-inverting way to apply the medicine-loaded vapor barrier to the infected toe(s); another method is a rolling up way like a condom to apply the medicine-loaded vapor barrier to the infected toe(s) or finger(s).

The methods disclosed in this invention are to bath infected toes or fingers being treated under related medicine 24 hours a day without evaporating the anti-fungus medicine, even during your shower or walking.

The kit invented here includes the following materials which have been used in these methods: disposable nitrile glove, anti-fungus medicine such as Vicks Vapor Rub, and latex-free bandaging tape. Patients have to change the medicine and put it on with the same methods daily. For treatment time, it takes approximately the same it takes for a nail to grow a full length, approximately four (4) months and this could vary with the individuals.

The key features of this invention are stopping medicine evaporation and keeping some room for infected toes or fingers within the vapor barrier to bath the infected areas all the time (all day long) and completely. Also, the methods were used in this invention will isolate the infected toe(s) or finger(s) to contaminate other toe(s) or finger(s) for the same person or other persons in the common areas.

### BRIEF DESCRIPTION OF THE DRAWINGS

FIG. 1 is a perspective view of an index finger covered by the thumb section of an extra-large disposable nitrile glove.

FIG. 2 is a perspective view of an anti-fungus agent in a cream form is loading to the top of the thumb section of an extra-large disposable nitrile glove.

FIG. 3 is a perspective view of the anti-fungus loaded thumb section is removing off the index finger.

FIG. 4 is a perspective view of four small finger tips (two pinkies and two ring fingers, first digits only) are inserting into the nitrile thumb.

FIG. 5 is a perspective view of the four fingertips are inverting the nitrile thumb over the big toe.

FIG. 6 is a perspective view of the four fingertips are inverting and dragging the nitrile thumb over the big toe.

FIG. 7 is a perspective view of a latex-free bandaging tape is used to secure the nitrile thumb to the toe.

FIG. 8 is a perspective view of three components (condom-like vapor barrier, anti-fungus cream, and tape) in the kit to treat nail infection.

FIG. 9 is a top view of a condom-like vapor barrier and a sectional side view of a rolled up condom-like vapor barrier.

FIG. 10 is a perspective view of the anti-fungus cream is loading to the center of the condom-like vapor barrier.

FIG. 11 is a perspective view of the anti-fungus cream loaded condom-like vapor barrier applies to the big toe.

FIG. 12 is a perspective view of a latex-free bandaging tape is used to secure the nitrile thumb to the big toe.

FIG. 13 is a side view of the big toe taped with anti-fungus cream loaded condom-like vapor barrier.

### DETAILED DESCRIPTION OF THE PREFERRED EMBODIMENTS

The present invention disclosed methods and related kit to treat nail infection. The key point of this invention is to bath the infected nail under the medicine 24 hours without evaporating, which is also the most effective way (if not the only way) to treat fungus infected nail with the current available tropical agents such as Vicks Vapor Rub. With the tight tape to secure the vapor barrier and anything inside, this invention prevent contamination in a common area such as shower place.

The following methods show the detailed steps to treat infected nail(s) under this invention:

A. Barrier-Inverting Way Method:

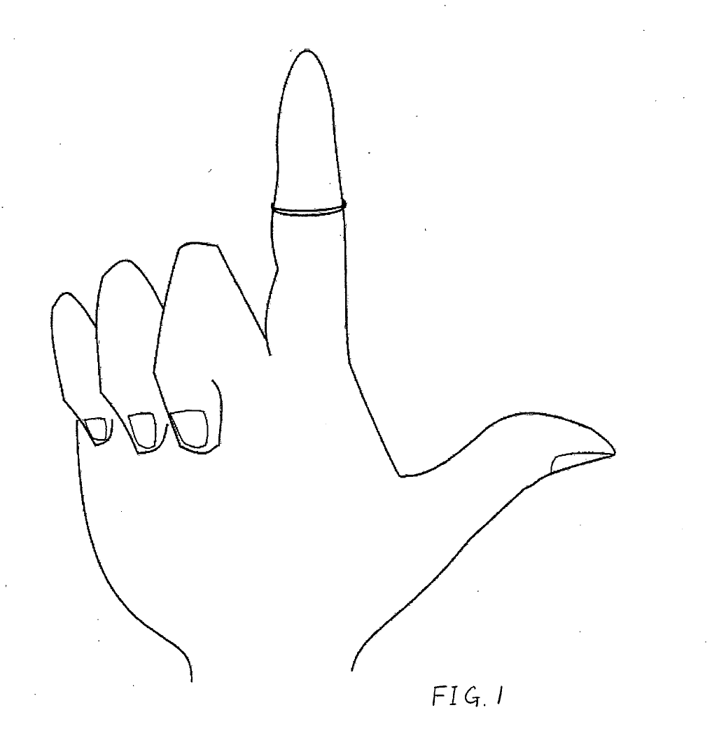

### Big Toe Application (see FIG. 1-7)

To treat toenail fungus with Vicks Vapor Rub, patients have to clean and trim your toenails, then wash them well and make sure they are completely dry.

### Preparing Vapor Barrier and Anti-Fungus Medicine:

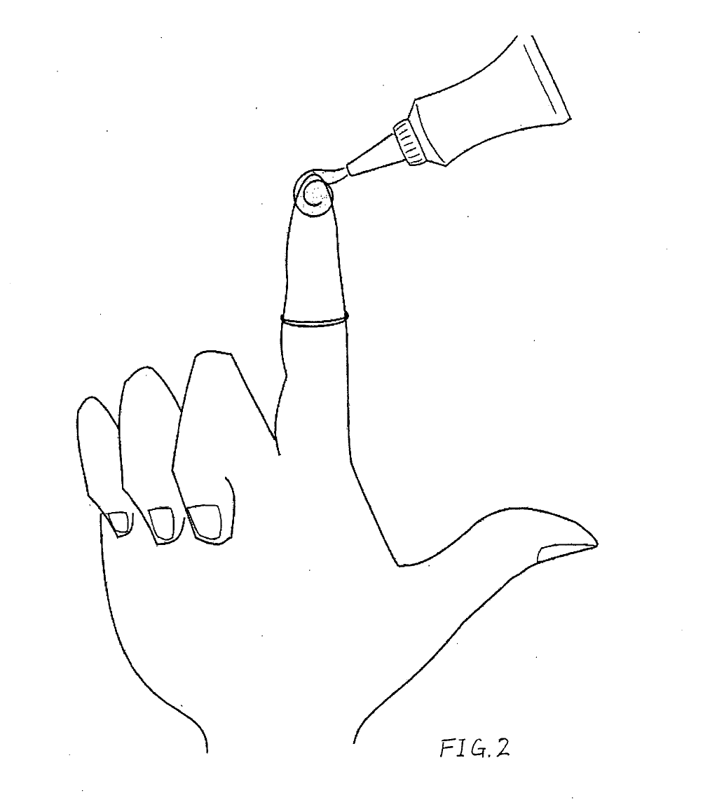

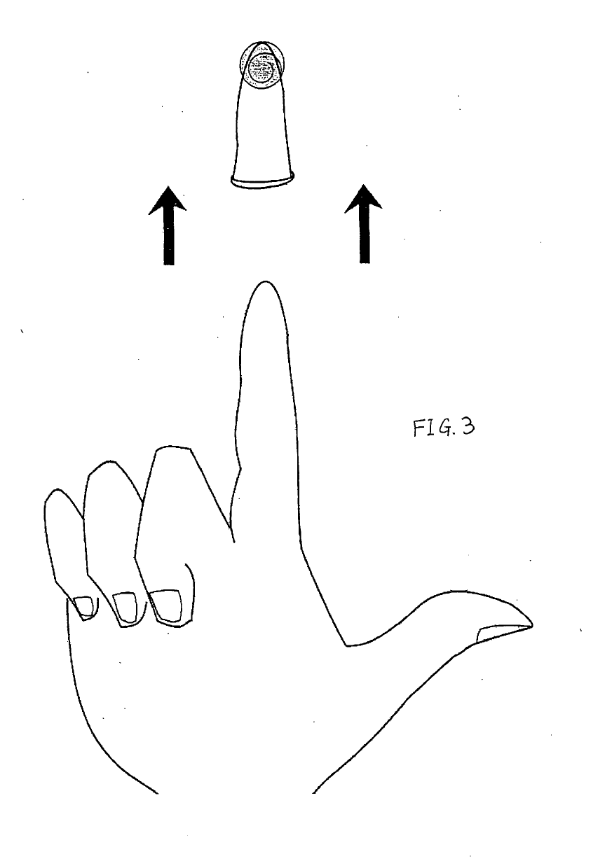

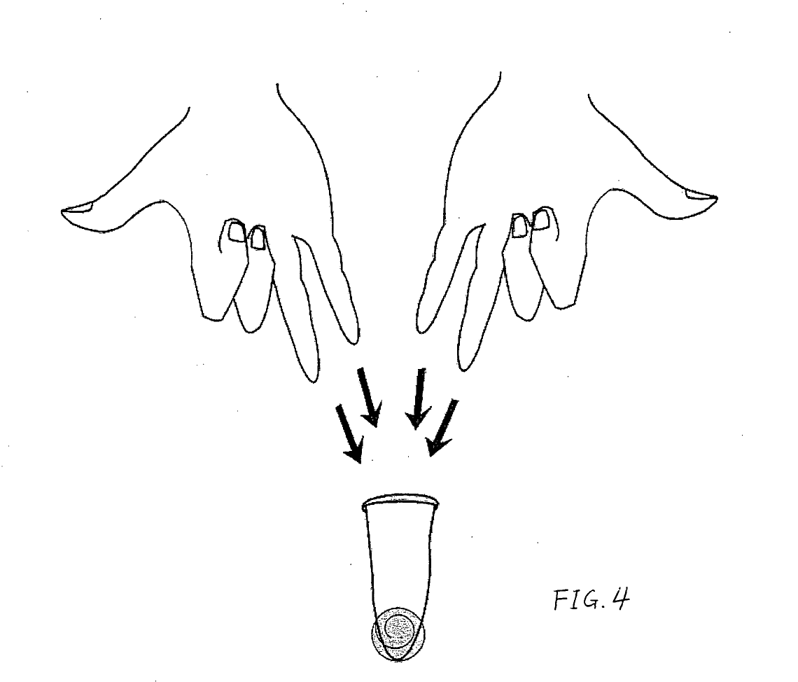

1. The Thumb of an extra-large disposable nitrile glove is cut off and the rest of the glove is set aside or disposed of The length of the retained nitrile thumb is approximately 1.5 to 1.625 inches in length.
2. The nitrile thumb (as a vapor barrier) is temporarily placed completely over the index finger (see FIG. 1).
3. The covered index finger is dipped into Vicks Vapor Rub and approximately ½ cc of Vicks is deposited onto the tip of the nitrile thumb (see FIG. 2).
4. The nitrile thumb is removed from the index finger and four small finger tips (two pinkies and two ring fingers, first digits only) are inserted into the nitrile thumb (see FIG. 3-4).

### Capping the Infected Big Toe:

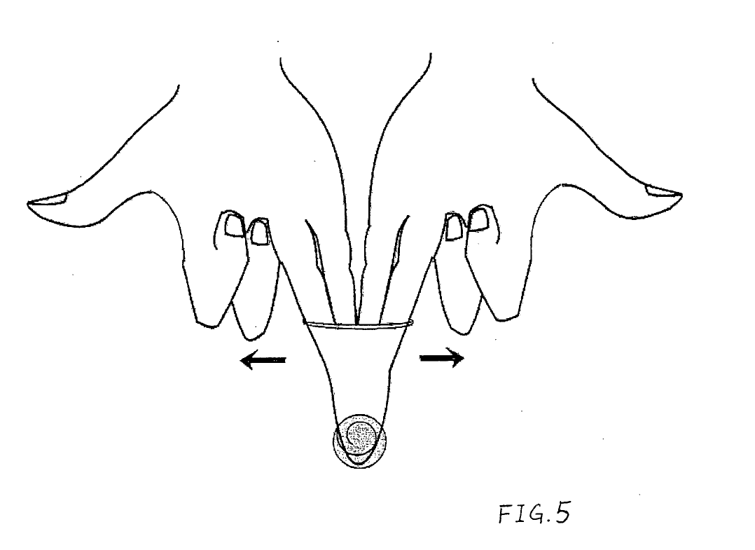

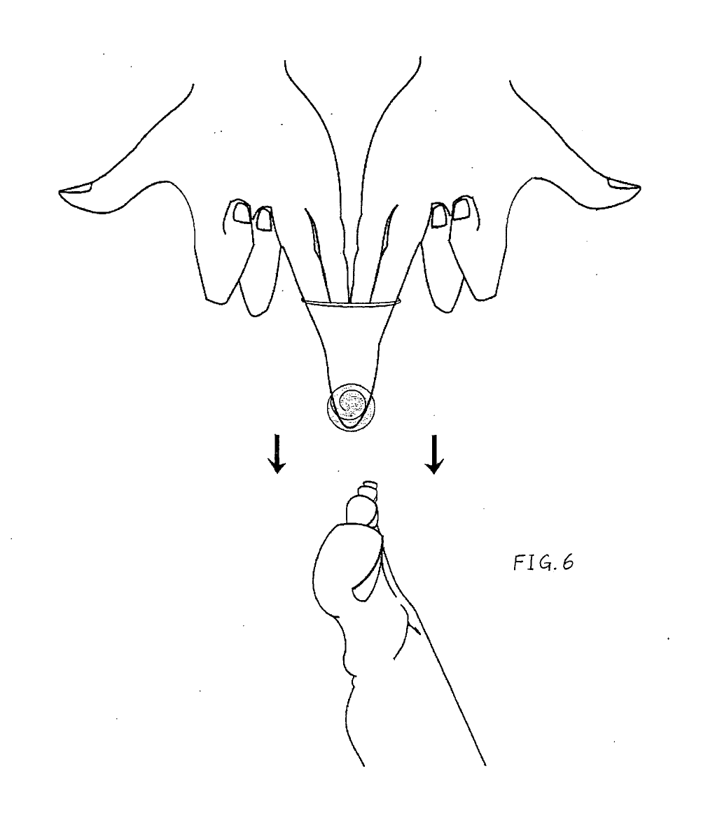

5. The Vicks is applied to the tip of the big toe as the four fingertips invert and drag the nitrile thumb over the big toe capturing the Vicks inside (see FIG. 5-6).
6. Any leakage of the Vicks (not trapped under the nitrile) is wiped away with tissue and alcohol so the tape will adhere.
7. The nitrile is not pulled fully over the toe (a small receptical space is left), so the trapped Vicks actually sloshes around the toe when walking and improves exposure.

### Taping:

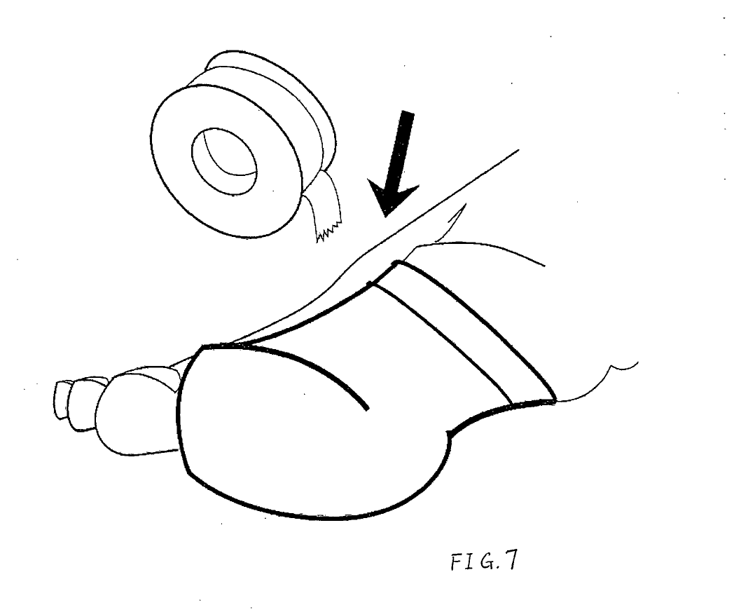

8. Latex free bandaging tape (one inch wide) is used to secure the nitrile thumb to the toe. The tape is applied snugly with approx. ½ inch overlapping the nitrile (see FIG. 7). Two layers of tape are used for strength. There is no Vicks under the tape. The tape bond is enhanced by pressing vigorously all around to maximize activation of the adhesive and protect the Vicks from rubbing off on to socks or something else. Leave it on and let the gel absorb into the nail. Repeat daily. The time it takes to notice results can be anywhere from 10 days to six weeks, depending upon how deep your fungus is.

### Smaller Toe Application:

With a cotton swab (Q-Tip, for example) apply Vicks directly to the infected nail. Cut one of the remaining fingers (approx. 1.0 inch length) off the Nitrile glove. (Any of the remaining fingers on the nitrile glove can be used for this). In this case the glove tip does NOT get inverted in such application: Pinch the tip of the nitrile finger-tip between thumb, index and middle fingers so that the cut end opens up. Slide the nitrile finger-tip over the Vicks-treated toe, then secure with the same taping method above as for big toe (with this method there should be no displacement of the Vicks into the taping surfaces and therefore no need for an alcohol wipe).

### Treatment Time:

This takes approximately the same it takes for a nail to grow a full length: approximately 4 months and this could vary with the individuals. Some of the infected nail may delaminate from the nail bed during treatment and should be clipped away to maximize exposure. Provided the vapor barrier is maintained (nail bed is kept moist), the nail should grow back to the length it was prior to infection. Moreover, the patients have to change the medicine and re-tape the vapor barrier daily.

B. Rolling Up Way Method:

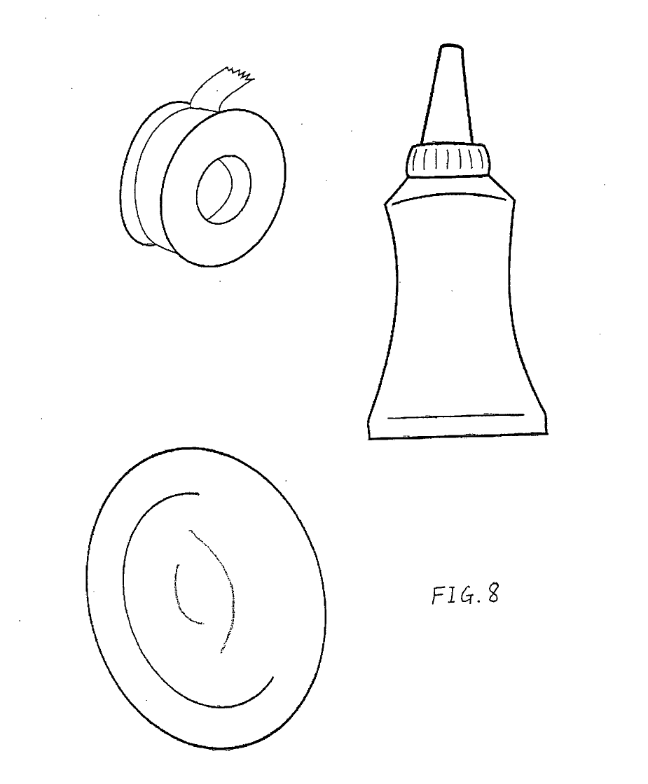

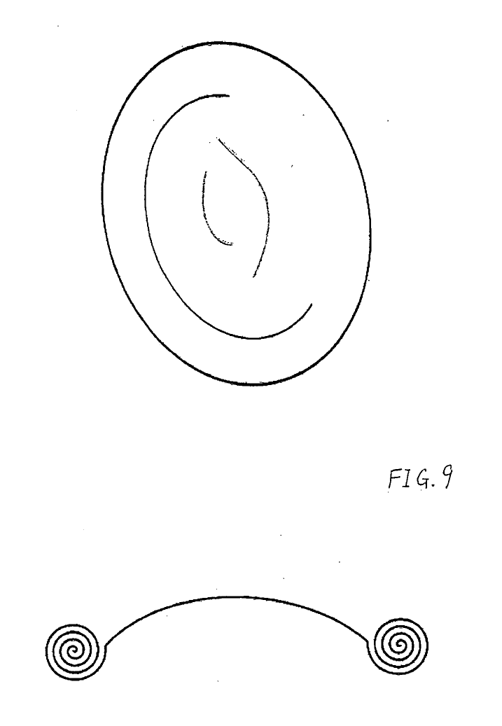

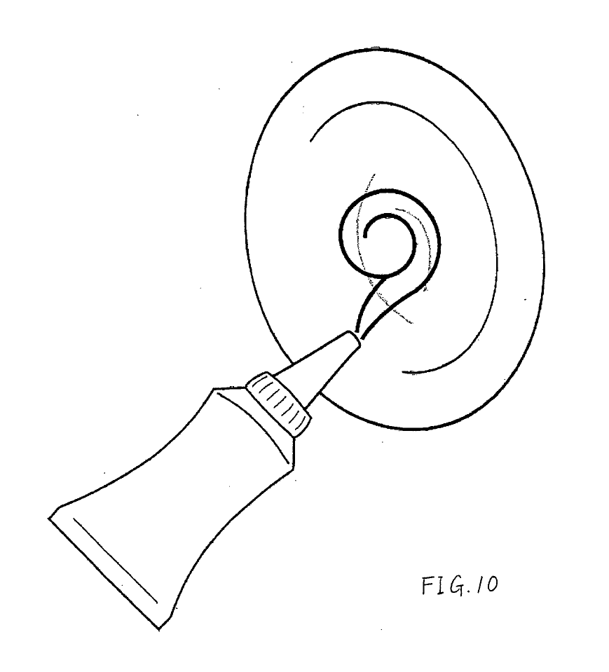

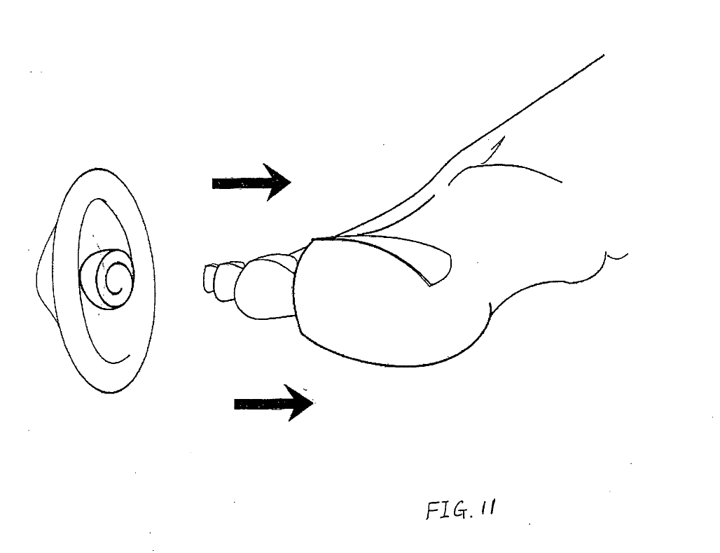

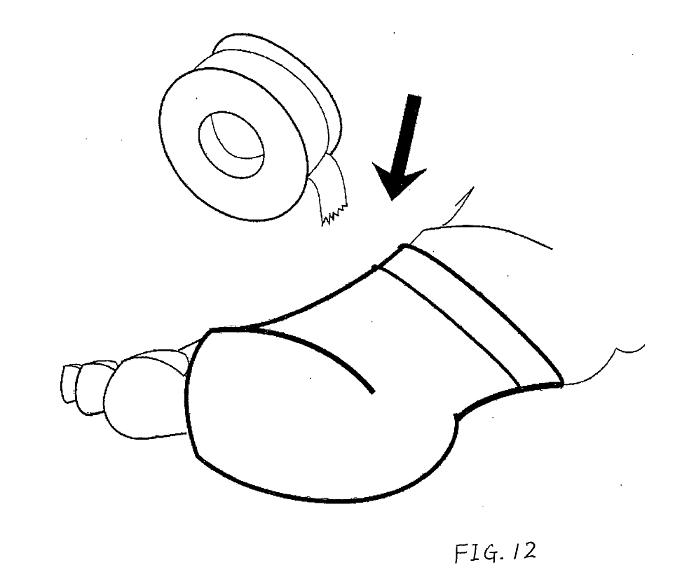

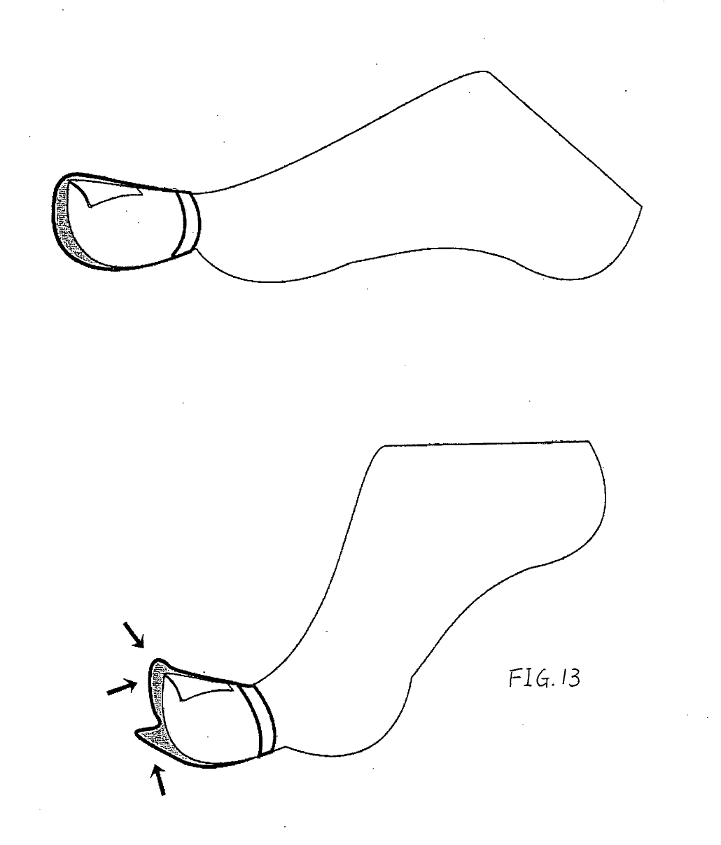

Another easier method (see FIG. 8-13) is to make a rolling up toe boot like a condom as a vapor barrier and not quite all the way down, so the Vicks has a captive pocket to stay when pressed onto the toe. The controllable amount of Vicks can be applied to the center of the rolled up toe boot. Ideally, there is a “fill-to” marking line inside the toe boot tip to make sure the amount of medicine applied.

This method also guarantees that no Vicks contamination on the taping area of the boot and on the toe when the boot is rolled up. A critical feature of this method is the fact that the toe boot is not skin tight (other than at the tape boundary after taping). The reason is that losing fit around the nail where the captured Vicks resides can and will slosh around the infected nail with every step walking to enhance exposure.

Also, thousands of people suffer with fingernail infection, often it has spread to all fingers and it affects their ability to get employment. So smaller flesh-colored fingertip boots with flesh-colored tape covers can be used to treat the fingernails as well.

The above described methods and the kit to treat nail fungus infection have the following advantages:
1. Easy to apply without doctors or any complicated equipment or device;
2. Prevent the cross-contamination among toes or fingers or among people;
3. All the components in the kit are commonly available from market and inexpensive;
4. The infected nails get treated all day long, even during the walking;
5. The medicine will not be evaporated under the vapor barrier, which increases the efficacy of the medicine;
6. The methods can be applied to both infected toes and infected fingers;
7. The unique gap between toe and vapor barrier allows medicine slosh around the infected nail with every step walking to enhance exposure;
8. The kit is small and easy to carry during travel time.

In summary, the present invention provides methods and a novel, portable and personalized anti-fungus kit, which is inexpensive and easy to use. Such methods improved the treatment significantly and can be used in the shower and even walking. This invention maximizes the medicine exposure to the infected area.

Although such anti-fungus kit and the methods of using the same according to the present invention has been described in the foregoing specification with considerable details, it is to be understood that modifications may be made to the invention which do not exceed the scope of the appended claims and modified forms of the present invention done by others skilled in the art to which the invention pertains will be considered infringements of this invention when those modified forms fall within the claimed scope of this invention.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. A kit for the treatment of a nail infection comprises: a disposable nitrile glove or a condom or the like which prevents evaporation, a anti-fungus medicine or an antibiotic, and a tape. Wherein said anti-fungus medicine or antibiotic is applied to infected nail(s) located in a toe or a finger, wherein said nitrile glove or a condom is put over infected toe(s) or finger(s) after applying said anti-fungus medicine or antibiotic, wherein a thumb or a finger of an extra-large said disposable nitrile glove is used to cover a toe or a finger, wherein said condom or the like is able to be rolled up, wherein said tape is used to secure said anti-fungus medicine or antibiotic inside the thumb or the finger of said disposable nitrile glove or said condom which is covered to an infected toe or an infected finger.

12. (canceled)

13. (canceled)

14. (canceled)

15. A kit of claim 11, wherein said anti-fungus medicine consists of camphor, eucalyptus oil, and menthol.

16. A kit of claim 11, wherein said tape is latex free bandaging tape.
